(19) United States
(12) Patent Application Publication
(10) Pub. No.: US 2017/0074843 A1
(43) Pub. Date: Mar. 16, 2017

(54) Thermal Ribbon Analysis

(71) Applicant: Quantum Signal LLC, Saline, MI (US)

(72) Inventors: David B. Johnson, Ann Arbor, MI (US); Robert M. Lupa, Vernon, IL (US); Kevin D. Melotti, Ann Arbor, MI (US); Mitchell M. Rohde, Saline, MI (US); Steven M. Rohde, Ann Arbor, MI (US); Christopher M. Showers, Royal Oak, MI (US); Michael G. Ploof, Ann Arbor, MI (US)

(21) Appl. No.: 15/360,125

(22) Filed: Nov. 23, 2016

Related U.S. Application Data
(63) Continuation of application No. 14/183,239, filed on Feb. 18, 2014, now abandoned.
(60) Provisional application No. 61/766,104, filed on Feb. 18, 2013.

(51) Int. Cl. G01N 33/00; B41M 7/00

(52) U.S. Cl. CPC G01N 33/00; B41M 7/009

(57) ABSTRACT

Thermal ribbon analysis is achieved via various approaches.

### RELATED APPLICATIONS

The present patent application is a continuation of the non-provisional patent application filed on Feb. 18, 2014, and assigned application Ser. No. 14/183,239, which itself claims priority to the previously filed and presently pending provisional patent application of the same title, filed on Feb. 18, 2013, and assigned patent application No. 61/766,104, which is hereby incorporated by reference.

### BACKGROUND

Visual odometry is the process of estimating or measuring the motion of a vehicle through analysis of image sequences collected by a camera mounted on or of the vehicle.

### DETAILED DESCRIPTION

Thermal ribbon analysis is a forensic-focused desktop device that permits fast, efficient, and automated recovery of data on thermal mass transfer or dye diffusion thermal transfer ribbons.

We claim:

1. A thermal ribbon analysis approach.

2. A system and method for thermal ribbon analysis.
